(19) United States
(12) Patent Application Publication
(10) Pub. No.: US 2022/0409115 A1
(43) Pub. Date: Dec. 29, 2022

(54) APPARATUS AND METHOD FOR MAGNETOENCEPHALOGRAPHY WITH ELECTROPERMANENT MAGNET ARRAY

(71) Applicant: Weinberg Medical Physics Inc, North Bethesda, MD (US)

(72) Inventor: Irving WEINBERG, North Bethesda, MD (US)

(21) Appl. No.: 17/853,051

(22) Filed: Jun. 29, 2022

Related U.S. Application Data
(60) Provisional application No. 63/216,196, filed on Jun. 29, 2021.

(51) Int. Cl. A61B 5/245; A61B 5/055

(52) U.S. Cl. CPC A61B 5/245; A61B 5/055; A61B 2562/0223

(57) ABSTRACT

Apparatuses and method are provided for collecting information for imaging. The apparatus has an array of two or more electropermanent magnets, and one or more limited magnetic sensors. The array and limited magnetic sensor are configured to be positioned in a vicinity of a region of interest,

### PRIORITY

This application claims priority to United States Provisional Patent Application Ser. No. 63,216,196, entitled “APPARATUS AND METHOD FOR MAGNETOENCEPHALOGRAPHY WITH ELECTROPERMANENT MAGNET ARRAY,” filed 29 Jun. 2021, the entirety of which is incorporated by reference.

### FIELD

Disclosed embodiments relate to apparatuses and methods for magnetoencephalography, in particular apparatuses and methods used in medical and veterinary applications.

### BACKGROUND

Magnetoencephalography (MEG) often utilizes sensors of magnetic fields that operate most effectively at low magnetic fields (i.e., less than one microTesla), or within a limited range of magnetic fields or frequencies of magnetic fields. Examples of sensor types with limitations in maximal ambient magnetic field include optically pumped magnetometers (OPMs) and superconducting quantum interference devices (“SQUID”). Magnetic sensors with limitations as to maximal ambient magnetic fields are described herein as “limited magnetic sensors”. As a result of such limitations, many MEG facilities employ magnetically-shielded rooms to reduce the ambient magnetic field.

### SUMMARY

Disclosed embodiments describe how arrays of electropermanent magnets (EPMs) may be used to provide magnetic field environments for limited magnetic sensors that operate most effectively in narrow regimes of magnetic field magnitude and/or frequency. The information collected from such sensors may be used to form images for MRI, MEG, conductivity imaging, magnetic particle imaging, or combinations of these types of images. For the purpose of this disclosure, the terms “magnetoencephalogram” and “magnetoencephalography” and “MEG” as used herein include all methods of assessing the voltages or currents or current pathways detected from a living organism, whether human or not.

### BRIEF DESCRIPTION OF FIGURES:

Aspects and features of the disclosed embodiments are described in connection with various figures, in which:

FIG. 1 illustrates an embodiment of an apparatus, including electropermanent magnets, in an array around a body part;

FIG. 2 illustrates an embodiment of the method according to the disclosed embodiments; and

FIG. 3 is a block diagram of a control system and power source according to the disclosed embodiments.

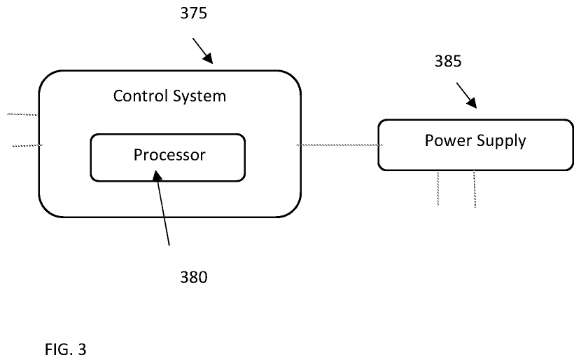

### DETAILED DESCRIPTION

The present invention will now be described in connection with one or more embodiments. It is intended for the embodiments to be representative of the invention and not limiting of the scope of the invention. The invention is intended to encompass equivalents and variations, as should be appreciated by those skilled in the art.

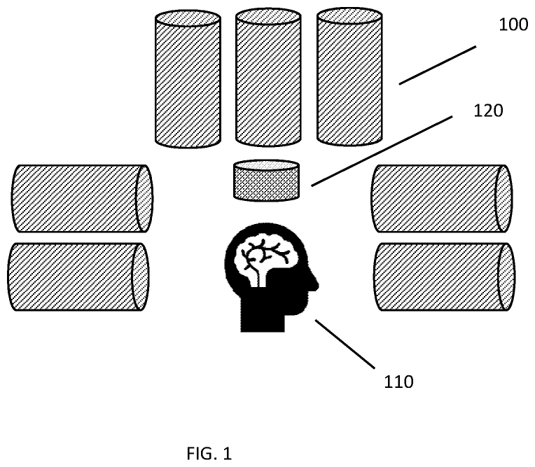

Disclosed embodiments describe an apparatus and method for sensing magnetic fields. FIG. 1 illustrates an embodiment of the apparatus, including electropermanent magnets 100 in an array around a body part of a living human or other living animal or of some other object in a region-of-interest 110, and one or more limited magnetic sensors 120. Electropermanent magnets may consist of magnetizable material, for example, AlNiCo rods, that may be magnetized by a current-carrying wire or wire coil as described, for example, in WEINBERG. U.S. Ser. No. 17/841,614, “APPARATUS AND METHOD FOR MAGNETIC RESONANCE IMAGING WITH ELECTROPERMANENT MAGNETS,” incorporated by reference in its entirety. Other means of activating or changing the magnetization of the magnetizable material may be used. In the disclosed embodiments, the magnetizable material stays at this magnetic state after the means of activating is no longer imposed.

The one or more limited magnetic sensors 120 may be an optically pumped magnetometer (OPM), or nitrogen vacancy magnetometer, or acoustically driven ferromagnetic resonance magnetometer, or other type of magnetometer that has limited sensitivity outside of a range of magnetic field magnitudes and/or frequencies. OPMs may have limited magnetic field magnitude or frequency ranges in which they operate, for example they may only operate between 0 and 1 nanoTesla, or with a 100 Hz bandwidth centered around some central frequency that may be tunable. Although the terms optical magnetic sensor and OPM is used, it is understood that the same principles of this invention may apply to other sensors of magnetic fields that operate most effectively at low magnetic field strengths and/or with very narrow frequency bandwidths (e.g. SQUID magnetometers, MEMS magnetometers, multiferroic magnetometers), and the terms OPM, OPMs, and “limited magnetic sensor” and “magnetic sensors” and “narrow regime magnetic sensor” are used in this disclosure to include such sensors.

It is known that to accommodate these magnetic sensor limits, MEG systems may include passive or active magnetic shielding in the walls of the room surrounding the MEG instrumentation to reduce (e.g. null) magnetic fields from nearby trains or other iron objects passing nearby. Passive shielding may include aluminum and/or ferromagnetic materials surrounding the MEG room. Active shielding may include large coils built into the room walls to null the Earth's field or the magnetic fields from other sources located outside the region of interest.

In the present disclosure, the magnetic fields created by the electropermanent magnet array 100 may achieve the intended result of nulling the magnetic field from sources outside the region of interest. This nulling may take place between pulse sequences intended to collect an MRI or magnetic particle image of objects providing anatomic and/or conductivity information in the region of interest, so that a composite or overlay anatomic/MEG image may be subsequently formed. Information from the anatomic and/or conductivity image so obtained may be useful in reconstructing the MEG image.

It is understood that the MEG image may be overlaid or otherwise integrated with an MRI generated with the aid of the electropermanent magnet array. It is understood that the role of the one or more limited magnetic sensors is to describe the magnetic fields generated or modulated by objects in the region of interest, whether those magnetic fields arise from physiological phenomena (e.g. neuronal firing) or from magnetized spins or other sources. It is understood that images or other data collected with the limited magnetic sensor may be used to guide or otherwise assist in procedures enabled by the EPMs, for example when manipulating one or more magnetic particles. It is understood that data collected with the limited magnetic sensor may be useful in detecting emanations of magnetic particles in the region of interest, for example in magnetic particle imaging, or when using spintronic particles or other emitters or modulators of magnetic fields.

Magnetic fields generated by the electropermanent magnet array may also assist in collecting useful information from the magnetic sensor 120 by spoiling magnetic field emanations from selected regions in the body part (“spoiled regions”) so that the emanations are outside the sensitive regime of the magnetic sensor (i.e. the unwanted regions). The spoiling may be implemented by having the EPM array create magnetic fields in these unwanted regions with frequencies that are outside the sensitive range for the magnetic sensor. Because of the non-linear conductivity of tissues, the frequency of emanations from these regions will be a combination of the imposed frequency (from the EPM array) and the intrinsic frequency of emanations from the tissues (e.g. from brain impulses), so that the magnetic sensor may be insensitive to emanations from these spoiled regions. Effectively, this means that the limited magnetic sensor will only be sensitive to regions in the body part that are not spoiled. It is understood that this process of accommodation of the limited magnetic sensor can be considered as a matching process, a term that is commonly used when configuring a system of sensors and amplifiers.

Advantages for MEG are that potentially a reduced number of limited magnetic sensors need to be employed to enable reconstruction, since reconstruction of the image of magnetic fields from desired regions is simpler when the limited magnetic sensor is only sensitive to one region at a time.

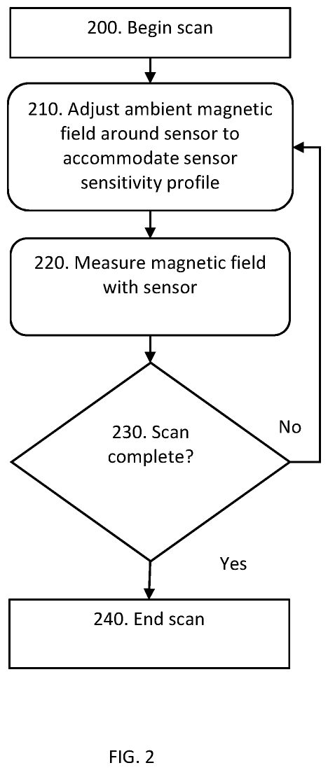

It is understood that the limited magnetic sensor may be used also to collect information about the spin state of objects in a region of interest, the information which may be useful in reconstructing the MRI. In such embodiment, one or more limited magnetic sensors may be used to collect information (e.g. for MRI) about the net magnetization of one or more regions of a body part by altering the precession frequency of spins in those regions. This alteration can be effected by using the EPM array to generate a magnetic field in the one or more regions that is associated (by the gyromagnetic ratio) with the frequency. The magnetic sensor's central frequency can be altered (see for example the 2017 paper by I Savukov et al, in Meas. Sci. Technol. DOI: 10.1088/1361-6501/aa58b4) so that it is most sensitive to the precession frequency in the regions. The tuning may be accomplished by configuring one or more EPMs near (e.g. less than 100 cm from) the magnetic sensor. This operation is included in FIG. 2, which may include a pulse sequence that is used for MRI, possibly in addition to collection of data for MEG.

FIG. 2 illustrates an embodiment of the method of operation of the disclosed embodiments. In operation 200 a scan is begun, for example by applying magnetic fields with the electropermanent magnet array 100 or by applying radiofrequency (RF) energy to spins in a region of interest in order to create an excited state. The magnetic field in the region of the limited magnetic sensor may be adjusted 210 by the electropermanent magnet array 100 to accommodate the limited magnetic sensor's magnetic sensitivity profile (i.e., limited in amplitude or frequency). For example, if the prescriber of the scan wishes to collect information about brain activity from a brain in the region-of-interest 110 in the vicinity of the one or more limited magnetic sensors (e.g. within one meter of the one or more limited magnetic sensors), then the magnetic field in the vicinity (e.g., within 100 cm) of the one or more limited magnetic sensors may be adjusted to zero or near zero by canceling the earth's magnetic field. The limited magnetic sensor may collect information related to the magnetic field emitted by the body part of interest 220, for example to collect data that may be reconstructed into a magnetoencephalography image. A controller such as a computer may determine if sufficient data have been collected to form the desired image or other data endpoint 230. If enough data have been collected, then operation 240 is attained and the scan is completed and the image may be formed. If not, then operations 210-230 may be repeated. Disclosed embodiments may be controlled by, for example, a controller or control system 375 in wired or wireless communications with the assembly and having a processor 380 and power supply 385.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as, for example, Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for collecting information for imaging comprising:
an array of two or more electropermanent magnets, and
one or more limited magnetic sensors configured to be positioned in a vicinity of a region of interest,
wherein signals from the one or more limited magnetic sensors are configured to be used to form an image describing magnetic fields generated or modulated by one or more objects in the region-of-interest.

2. The apparatus of claim 1, wherein a magnetic field generated by the array reduces a magnetic field arising from locations outside the region of interest to accommodate the limited sensitivity range of one or more limited magnetic sensors.

3. The apparatus of claim 1, wherein a magnetic field generated by the array alters the frequency of a magnetic field arising from locations outside the region of interest to accommodate the limited sensitivity range of one or more limited magnetic sensors.

4. The apparatus of claim 1, wherein the one or more limited magnetic sensors comprises a magnetometer that has limited sensitivity outside of a range of magnetic field magnitudes and/or frequencies

5. The apparatus of claim 1, wherein the one or more limited magnetic sensors comprises an optically pumped magnetometer (OPM), an nitrogen vacancy magnetometer, or an acoustically driven ferromagnetic resonance magnetometer

6. A method of collecting information for imaging comprising:
positioning an array of two or more electropermanent magnets and one or more limited magnetic sensors in the vicinity of a region of interest, and
using signals from the one or more limited magnetic sensors to collectinformation for generating an image describing the magnetic field generated or modulated by one or more objects in the region-of-interest.

7. The method of claim 6, further comprising generating a magnetic field by the array to reduce the magnetic field arising from locations outside the region of interest to accommodate the limited sensitivity range of one or more limited magnetic sensors.

8. The method of claim 6, further comprising generating a magnetic field by the array to alter the frequency of the magnetic field arising from locations outside the region of interest to accommodate the limited sensitivity range of one or more limited magnetic sensors.

9. The method of claim 6, where the image is a magnetoencephalogram.

10. The method of claim 6, where the image is a magnetic resonance image.

11. The method of claim 6, where the image is a magnetic particle image.

12. The method of claim 6, where the image is used to guide a procedure enabled by the array of two or more electropermanent magnets.

13. The method of claim 6, wherein the one or more limited magnetic sensors are used to describe magnetic fields emitted or modulated by particles in the region of interest.

14. The method of claim 6, wherein the one or more limited magnetic sensors comprises a magnetometer that has limited sensitivity outside of a range of magnetic field magnitudes and/or frequencies

15. The method of claim 6, wherein the one or more limited magnetic sensors comprises an optically pumped magnetometer (OPM), an nitrogen vacancy magnetometer, or an acoustically driven ferromagnetic resonance magnetometer.
